(19) United States
(12) Patent Application Publication
(10) Pub. No.: US 2021/0322277 A1
(43) Pub. Date: Oct. 21, 2021

(54) SIMULATED FLOW-CONTROL BREAST FEEDING SYSTEM

(71) Applicant: Little Latchers, LLC, Brooklyn, NY (US)

(72) Inventor: Kimberly Meikle, Brooklyn, NY (US)

(21) Appl. No.: 17/364,883

(22) Filed: Jul. 1, 2021

Related U.S. Application Data
(63) Continuation-in-part of application No. 15/933,302, filed on Mar. 22, 2018, now Pat. No. 11,076,969.
(60) Provisional application No. 62/543,150, filed on Aug. 23, 2017.

(51) Int. Cl. A61J 11/00; A61F 2/52; G16H 40/63

(52) U.S. Cl. CPC A61J 11/001; A61F 2/52; A61J 11/005; A61J 1/10; G16H 40/63; A61F 2002/526; A61J 2200/70; A61J 11/0005

(57) ABSTRACT

System comprising of a simulating flow control breastfeeding where a tube linking a refillable housing chamber to a prosthetic nipple, an analyzing module linked to a sensor to determine a physical, physiological, or emotional state of the infant/user by mapping the received input parameter with a pre-stored set of parameters from the parameter store via the analyzing module and dynamically interact with the infant/user based on a determined output to promote a non-latching simulating breastfeeding experience to prevent bruised, sore nipples or engorged breasts.

### CROSS-REFERENCE TO RELATED APPLICATION

This application claims priority to U.S. Provisional Patent Application Ser. No. 62/653,150 filed on Aug. 23, 2017 which is incorporated herein in its entirety.

### FIELD OF THE INVENTION

The present invention relates to a simulated breastfeeding device for effective nursing of infants. More particularly, the invention relates to a system for enabling a parent to nurse an infant in a safe and efficient manner.

### BACKGROUND OF THE INVENTION

There is a tremendous psychological and emotional benefit that comes along with breastfeeding that aids in infants' development that they do not get from bottle feeding alone, in addition to the psychological and emotional benefit to the parent of bonding with the child. However, there are many instances in which parents are unable to breastfeed their babies. For instance, women who have had mastectomies may be unable to breastfeed, and some diseases or medications require women to forego breastfeeding for the safety of their child. Adoptive parents of newborn babies also do not have the ability to breastfeed. Thus, many parents miss out on the bonding experience of breastfeeding their infant, and their infants do not receive the benefits of bonding with their mother.

In many cases, the time taken to establish milk supply ranges between four to six weeks after a baby's birth. This also requires the mother to breastfeed the infant continuously, around the clock. Such continued breastfeeding where the infant remains in constant contact with the mother's breast and nipples could, in many instances, result in rashes, irritability caused by the swelling of the nerves, and bleeding around the nipples or nerves due to incorrect latching by the infant. The World Health Organization estimated that approximately 60% of mothers who stopped breastfeeding did so earlier than desired and the early termination was positively associated with mother's concerns regarding difficulties with lactation, swollen breasts, irritability, rashes, etc. There are no products in the market that facilitate latch free or contactless nursing: simulated breast-feeding obviating the need of mouth-nipple contact, thereby avoiding or mitigating rashes, irritability, or swelling in the nipple region of the mother. This void, in turn, results in mothers giving up on the chance to breastfeed and resort to only formula feeding or exclusive pumping. More importantly, the market is void of any products that take into account the infants' physical, emotional and physiological state and determine an output to promote a non-latching simulating breastfeeding experience to prevent bruised, sore nipples or engorged breasts.

Other devices have been proposed to solve this problem, such as wearable baby bottles that can strap to the chest. These solutions can be clunky and unnatural and take away the skin-to-skin connection of natural breastfeeding. The simulated breast-feeding product from Medela include, a compact disposable device which provides a suckling infant with a liquid diet supplement simultaneously with normal breastfeeding. However, it still requires direct contact with the mother's breasts and hence does not address the irritability and swollen nerves issue that prolonged breastfeeding tends to cause in mothers.

### SUMMARY OF THE INVENTION

In an embodiment of the invention, the breastfeeding device simulates natural breastfeeding experience and delivers fluid, milk or infant formula to the nursing infant in a contactless or latch free manner than any prior art in this field. This device integrates physical sensors to the breastfeeding device and includes a neck strap, refillable housing chamber that contains fluid, a tubing that connects the housing chamber to a prosthetic nipple via a sealing attachment and a prosthetic nipple for infant latching to nurse. The invention is a notable step forward in creating an effective, safe and contactless nursing which mitigates and prevents bruised, sore nipples or engorged breasts.

More specifically, the present invention relates to a system to breastfeed an infant to nurse in a safe and effective manner. In an embodiment, the device includes a neck strap, an attachable refillable housing chamber with a sealing contraption at one end, a tube linking the refillable housing chamber to a prosthetic nipple. Further yet, in an embodiment of the invention, the prosthetic nipple of the system comprises of an opening to deliver the fluid from the refillable housing chamber to an infant to promote a non-latching simulating breastfeeding experience. Additionally, in an embodiment of the invention, the device may include a sensor, a network interface, a processor, an analyzing module, a non-transitory storage element with encoded instructions coupled to the processor configure the system to receive an input parameter from an infant or user, determine a physical, physiological or emotional state of the infant/user via the analyzing module and dynamically interact with the infant/user based on a determined output.

The sensor activity of the system corresponds to at least one of aspects and advantages of this invention and may be realized in other applications, aside from the intended application of nursing/feeding an infant. Examples of other pertinent applications that may exploit the aspects and advantages of this invention include, but are not limited to, monitor the life and volume of fluid consumed by the infant during the feeding time, to monitor if the infant falls asleep while nursing, or if the infant is feeding for comfort or hunger. The device could also be used to sense infant temperature to trigger an alarm when it detects an abnormality in respiration rhythms or abnormal body temperatures.

The invention will typically employ one or more sensors, one or more processors (e.g. compute processors such as microprocessors, and the like), a network interface, an analyzing module to analyze and monitor the characteristics of infant nursing and feeding. These characteristics include feeding, latching, sleeping, crying, burping and the like. The invention may use data from these various sensors and processors to establish a baseline of what is “normal” for these types of activities. These baselines may be established across various characteristics and/or customized for each infant to establish an infant profile. These baselines may also be adjusted for different situations that may be possible outcomes.

### BRIEF DESCRIPTION OF DRAWINGS

FIG. 1 describes an exemplary embodiment of the invention.

FIG. 2 illustrates an exemplary illustration with various embodiments of the disclosure that can be practiced.

FIG. 3 illustrates an exemplary illustration of one of the embodiments invention.

FIG. 4 illustrates an exemplary embodiment of the invention.

FIG. 5 illustrates a method flow of the invention.

FIG. 6 illustrates a network diagram of one embodiment of the invention.

FIG. 7 illustrates a block diagram of one embodiment of the invention.

FIG. 8 illustrates an exemplary illustration of one of the embodiments invention.

FIG. 9 illustrates an exemplary illustration of one of the embodiments invention.

FIG. 10 illustrates an exemplary illustration of one of the embodiments invention.

FIG. 11A illustrates an exemplary illustration of one of the embodiments invention.

FIG. 11B illustrates an exemplary illustration of one of the embodiments invention.

### DETAILED DESCRIPTION OF DRAWINGS

The present invention will now be described more fully with reference to the accompanying drawings, in which embodiments of the invention are shown. However, this disclosure should not be construed as limited to the embodiments set forth herein. Rather, these embodiments are provided so that this disclosure will be thorough and complete, and will fully convey the scope of the disclosure to those skilled in the art. Like numbers refer to like elements throughout.

In the following description, for purposes of explanation, numerous specific details are set forth in order to provide a thorough understanding of the invention. It will be apparent, however, to one skilled in the art that the invention can be practiced without these specific details.

Reference in this specification to “one embodiment” or “an embodiment” means that a feature, structure, or characteristic described in connection with the embodiment is included in at least one embodiment of the invention. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but no other embodiments.

### Overview:

The primary purpose of the disclosure is to simulate a contactless latch-free breastfeeding experience without causing rashes, irritability, pain or swelling to the mother's breast to ensure a safe nursing experience. This experience helps the parent transition effortlessly into the role of a new parent and simultaneously enjoy the bonding time.

### EXEMPLARY ENVIRONMENT

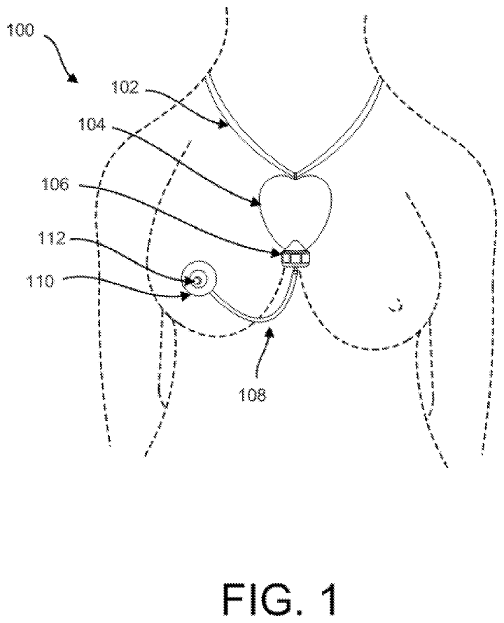

FIG. 1 illustrates an exemplary environment in which various embodiments of the present invention can be practiced. In an embodiment of the invention, a simulating breast-feeding system 100 comprises of a neck strap 102, an attachable refillable housing chamber 104 with a sealing contraption 106 at one end, a tube 108 linking the refillable housing chamber 104 to a prosthetic nipple 110 wherein, the prosthetic nipple 110 comprises of an opening 112 to deliver the fluid from the refillable housing chamber 104 to an infant to promote a non-latching simulating breastfeeding experience to prevent bruised, sore nipples or engorged breasts. Additionally, this system solves the problem of the lack of ability for communities such as, but not limited to, breast cancer survivors, LGBTQ, adoptive parents as well as family members to engage in the bonding breastfeeding experience with the infant.

Further yet, in an additional embodiment of the invention, the breast-feeding system may comprise of a sensor, a network interface, a processor, an analyzing module, a non-transitory storage element coupled to the processor and encoded instructions stored in the non-transitory storage element, wherein the encoded instructions when implemented by the processor, configure the system to receive an input parameter from an infant/user to determine a physical, physiological or emotional state of the infant/user by mapping the received input parameter with a pre-stored set of parameters via the analyzing module and dynamically interacting with the infant/user based on a determined output.

Additionally, in an embodiment of the invention, the system may further comprise of a sensor attached to at least one of, but not limited to, a sealing contraption, neck strap, prosthetic nipple, tubing or the refillable housing. To enable continuous monitoring, the user uses a sensor attached on any one of, but not limited to, the sealing contraption 106, neck strap 102, prosthetic nipple 110, tubing 108 or the refillable housing chamber 104 and a mobile communication device (not shown). The sealing contraption 106, neck strap 102, prosthetic nipple 110, tubing 108 or the refillable housing chamber 104 are typically embedded/equipped with one or more motion sensors, physiological sensors and environmental sensors. Examples of these sensors include, but are not limited to accelerometers, gyroscopes, inclinometers, geomagnetic sensors, global positioning systems, impact sensors, microphones, cameras, heart rate monitors, pulse oximeters, blood alcohol monitors, respiratory rate sensors, transdermal sensors, galvanic skin response (GSR) sensors and electromyography (EMG) sensors. In an embodiment of the present invention, the data captured by the one or more sensors is sent to the processing unit through the network. Further yet, the simulating breast-feeding system may comprise of a sensor to monitor fluid consumption to gauge if, the infant is feeding for comfort or hunger. Additionally, in another embodiment of the invention, the simulating breast-feeding system may comprise sensors to detect at least one of, but not limited to, infant temperature, heart rate, pulse rate, respiratory patterns or rhythms or accuracy of latching.

Further yet, the simulating system 100 may be worn on one or more body parts of the user, such as wrist, waist, neck, arm, leg, abdomen, chest, thigh, head, ear and fingers as well as attached to a plurality of attachments such as, but not limited to, an armband, headband, backpack worn by the user.

The mobile communication device is a portable device that has the capability of communicating over the network in real time, presenting periodic surveys to the user and receiving response from the infant/user on the periodic surveys. Examples of the mobile communication device include, but are not limited to, a smartphone, a tablet, a personal digital assistant (PDA) and a mobile phone.

In an embodiment of the present invention, the data captured by the one or more sensors of the simulating breast-feeding system 100 is first sent to the mobile communication device and thereby, sent to the processing unit over the network. The simulating breast-feeding system 100 communicates with the mobile communication device over a short range wireless communication medium. Examples of the short range wireless communication medium include Bluetooth, ZigBee, Infrared, Near Field Communication (NFC) and Radio-frequency identification (RFID).

The network may be any suitable wired network, wireless network, a combination of these or any other conventional network, without limiting the scope of the present invention. Few examples may include a LAN or wireless LAN connection, an Internet connection, a point-to-point connection, or other network connection and combinations thereof. The network may be any other type of network that is capable of transmitting or receiving data to/from host computers, personal devices, telephones, video/image capturing devices, video/image servers, or any other electronic devices. Further, the network is capable of transmitting/sending data between the mentioned devices. Additionally, the network may be a local, regional, or global communication network, for example, an enterprise telecommunication network, the Internet, a global mobile communication network, or any combination of similar networks. The network may be a combination of an enterprise network (or the Internet) and a cellular network, in which case, suitable systems and methods are employed to seamlessly communicate between the two networks. In such cases, a mobile switching gateway may be utilized to communicate with a computer network gateway to pass data between the two networks. The network may include any software, hardware, or computer applications that can provide a medium to exchange signals or data in any of the formats known in the art, related art, or developed later.

In an embodiment of the present invention, the processing unit receives the sensor data from the simulating breast-feeding system 100 and response on the periodic surveys from the user/infant. Thereby, the processing unit correlates the sensor data with the response on periodic surveys, generates reports corresponding to the physical, physiological or emotional state of the user/infant and sends the reports and other relevant data to the user. These reports enable the user to track/monitor the progression of feeding in the infant. In an embodiment of the present invention, the user is enabled to view the reports, as generated by the processing unit using one or more devices selected from the group comprising a smartphone, a computer, a laptop, a tablet, a personal digital assistant (PDA) and a mobile phone.

In an embodiment of the invention, the simulating breast-feeding system 100 is configured to receive an input parameter from an infant/user, a network interface, a processor, an analyzing module, a non-transitory storage element coupled to the processor with encoded instructions stored in the non-transitory storage element, wherein the encoded instructions when implemented by the processor configure the simulating breast-feeding system 100 to receive an input parameter from an infant/user, determine a physical, physiological or emotional state of the infant/user by mapping the received input parameter with a pre-stored set of parameters via the analyzing module and dynamically interacting with the infant/user based on a determined output. The pre-stored of parameters may be a plurality of infant behaviors as outlined in American academy of Pediatrics, World health organization, National Institute of child health and human development, Food and Drug administration, American academy of family physicians. Further yet, in another embodiment of the invention, the physical input parameter may be at least one of, but not limited to, motion, sleep, gestural, visual, audio or environmental factors/conditions. Moreover, the sleep related characteristics of the infant may be indicative of at least one of, duration of sleep time, number of times awake, sound sleep, light sleep and awake time. Additionally, the physiological input parameters may be at least one of, but not limited to, age, gender, race, medical history, heart rate, medication history, blood pressure, sweat, duration of feeding, number of feedings and food intake of user/infant.

Additionally, environmental conditions may affect infant activity. The environmental conditions can be at least one of, but not limited to, wind velocity, temperature, humidity, aridness, light, darkness, noise pollution, exposure to UV, airborne pollution and radioactivity. Further yet, data generated from a set of parameters corresponding to at least one of, but not limited to, user/infant reported symptoms and side effects, periodic surveys may be used to generate a personal profile of the infant/user. The data generated may in any one of, but not limited to, audio, video or an image input and further, implemented on at least one of, but not limited to a mobile communication device, body worn device, wearable device, tablet and or IoT.

Further yet, in an embodiment of the invention, a deviation from the personal profile may be alerted to the user by at least one of, a visual or color appearance, audio or sound, buzzer, vibration, fine motor skills, memory-based tasks, repeating actions, sounds or movements. Moreover, the simulating breast-feeding system 100 may comprise detection a deviation from the personal profile of the user/infant using machine learning algorithm.

Further yet, a machine learning algorithm may be employed to inform upstream processes. Additionally, in simulating breast-feeding system 100, the aggregation of the set of parameters occurs from at least one of, a mobile communication device, wearable device, smartphone, tablet, personal digital assistant (PDA) and Internet of Things device.

Further yet, in another embodiment, the simulating breast-feeding system 100 may further comprise integration with any one of a third-party application via an Application Program Interface (API) 207. This allows for 3rd party database integration, such as Electronic Medical Records (EMR), health monitoring, proxy health provisioning, remote server and, or a cloud-based server for other 207 downstream analytics and provisioning. Additionally, the completed automated responses may be saved onto a remote cloud-based server for easy access for data acquisition and archival analytics for future use.

In another embodiment of the invention, simulating breast-feeding system 100 may allow for easy saving, searching, printing, and sharing of completed automated response information with authorized participants. Additionally, simulating breast-feeding system 100 may allow for non-API applications, for example, building reports and updates, create dashboard alerts as well as sign in/verifications 207. Alternatively, sharing may be possible with less discrimination based on select privacy filters.

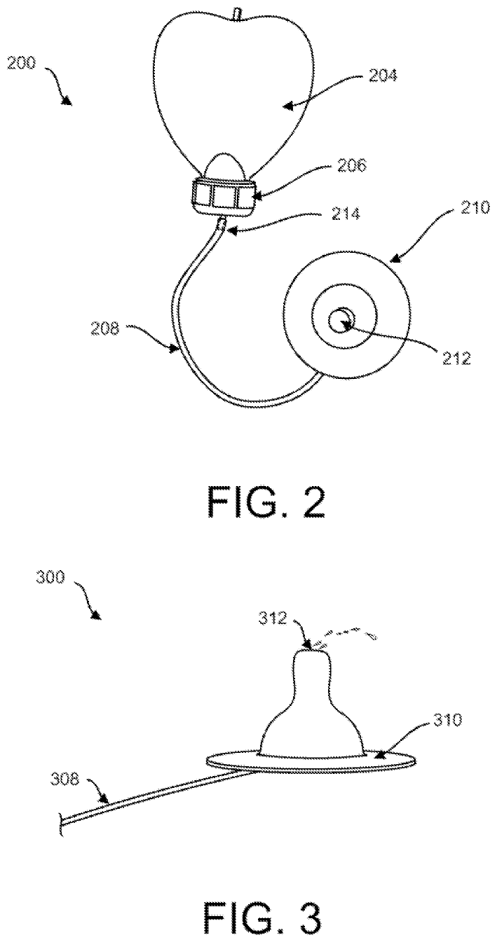

FIG. 2 illustrates a simulating breast-feeding apparatus. As shown in FIG. 2, a simulating breast-feeding apparatus 200 comprises of a neck strap (not shown), an attachable refillable housing chamber 204 with sealing contraption 206 at the one end and a tube 206 linking the refillable housing chamber 204 to a prosthetic nipple 210 wherein, the prosthetic nipple 210 comprises of an opening 212 to deliver the fluid from the refillable housing chamber 204 to an infant to promote a non-latching simulating breastfeeding experience to prevent bruised, sore nipples or engorged breasts.

In an embodiment of the invention, the housing chamber 204, prosthetic nipple 210 or the tubing 208 is at least one of, a plurality of shapes, sizes, colors or opaqueness. Further yet, the refillable housing chamber 204 may be insulated to maintain at least one of, hot, cold or room temperature for the fluid. Further yet, in an embodiment of the invention, the size of the prosthetic nipple 210 may be of varying sizes depending on at least one of, but not limited to, age, weight, gender, amount of food intake of the infant.

FIG. 3. illustrates a side angle view of the prosthetic nipple 300. As shown in FIG. 3, a tube 308 linking the refillable housing chamber (not shown) to a prosthetic nipple 310 wherein, the prosthetic nipple 310 comprises of an opening 312 to deliver the fluid from the refillable housing chamber (not shown) to an infant to promote a non-latching simulating breastfeeding experience to prevent bruised, sore nipples or engorged breasts.

Additionally, in an embodiment of the present invention, the prosthetic nipple 310 or tubing 308 comprises of, at least one of, naturally or man-made rubber, synthetic rubber, silicone or any plastic or synthetic polymer. Further yet, in an embodiment of the invention, the back end of the prosthetic nipple 310 maybe attachable to the skin via a self-adhering glue to establish a tight seal to prevent any leakage.

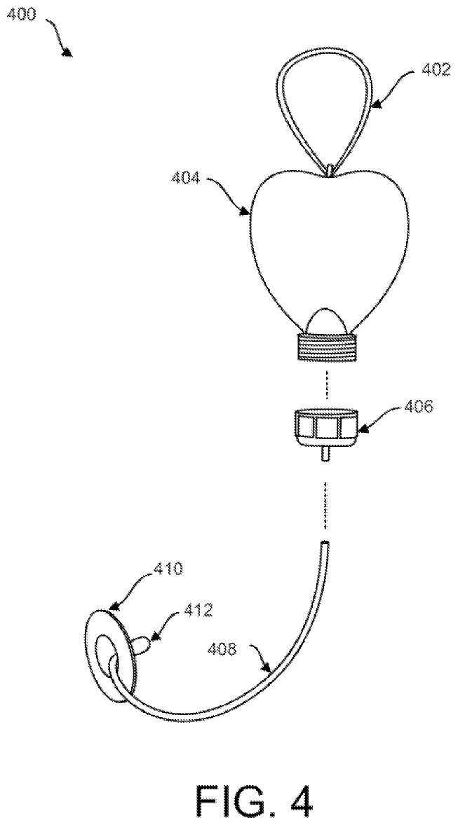

FIG. 4. shows an exemplary embodiment of the invention. As shown in FIG. 4, simulating breast-feeding apparatus 400 comprises of a neck strap 402, an attachable refillable housing chamber 404 with sealing contraption 406 at the one end and a tube 408 linking the refillable housing chamber 404 to a prosthetic nipple 410 wherein, the prosthetic nipple 410 comprises of an opening 412 to deliver the fluid from the refillable housing chamber 404 to an infant to promote a non-latching simulating breastfeeding experience to prevent bruised, sore nipples or engorged breasts.

In an embodiment of the invention, at least one of, neck strap, refillable housing chamber, sealing contraption, tubing or prosthetic nipple be modular. Further yet, the modular parts may be made of at least one of, non-GMO, non-toxic, organic-grade glue, BPA free or hypoallergic material. Additionally, the modular strap 402 comprises at least one of, nylon, polyester, silk, cotton, yarn, wool or fabric with a breakaway safety feature to prevent pulling or choking with or without a breakaway safety feature to prevent pulling or choking.

Further, in an alternative embodiment, the modular strap 402 may be worn as a waist belt or a shoulder strap that could be attached to a baby carrier or as an infant sling. Additionally, in an alternative embodiment of the invention, a clamp may be placed on the tubing 408 which may control fluid flow into the prosthetic nipple. Alternatively, a sensor may allow the user to stop the flow of fluid into the prosthetic nipple 410 by tapping on at least one of the modular parts. In yet another embodiment of the invention, the simulating breast-feeding apparatus may be held in any body worn garment or may be attached to a plurality of devices, for example, but not limited to, car seat, stroller, back-pack or sling.

In an alternative embodiment of the invention, the strap 402 may have a built-in chamber to hold the fluid connected to the prosthetic nipple 410. The tube 408 may be embedded into the strap. Additionally, the strap 402 may be a part of a back pack, a body worn garment such as, but not limited to, sports bra, vest, t-shirt, nursing bra, arm band, chest band, top etc.

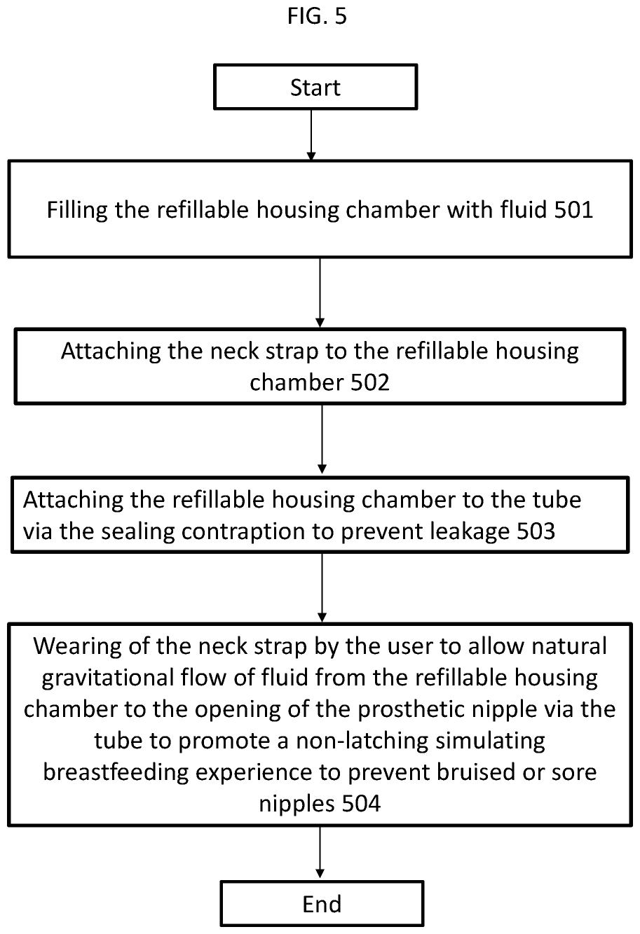

FIG. 5 shows a method for simulated breast-feeding system. The method comprises the steps of: (1) filling the refillable housing chamber with fluid 501, (2) attaching the neck strap to the refillable housing chamber 502, (3) attaching the refillable housing chamber to the tube via the sealing contraption to prevent leakage 503 and (4) wearing of the neck strap by the user to allow natural gravitational flow of fluid from the refillable housing chamber to the opening of the prosthetic nipple via the tube to promote a non-latching simulating breastfeeding experience to prevent bruised or sore nipples 504.

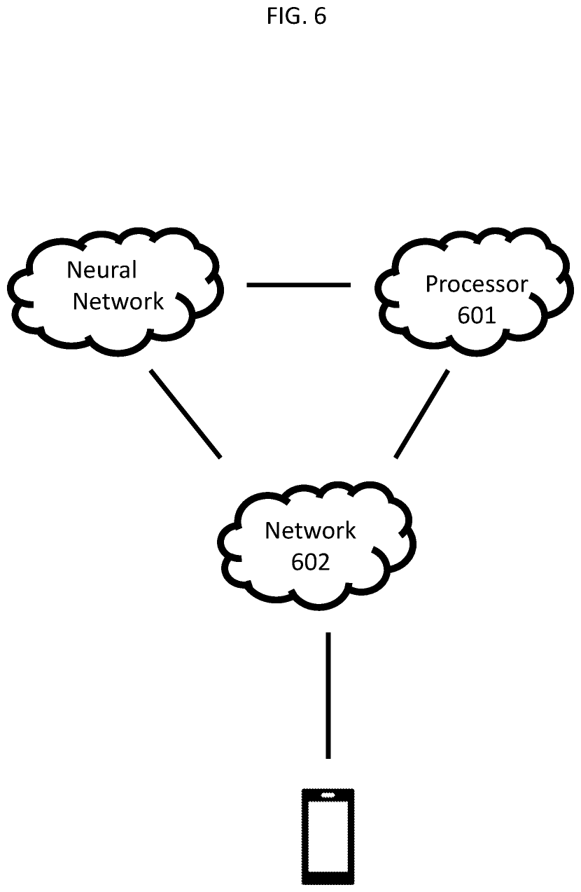

FIG. 6 an exemplary illustration of one of the embodiments invention. FIG. 6 illustrates a network diagram of the simulating flow-control breast-feeding system in accordance with an aspect of the invention. As shown, at least one processor 601 is connected to the Internet (network) 602 via either a wireless (e.g. WiFi link) or wired link to an Internet connected router, usually via firewall. The network 602 may be any class of wired or wireless network including any software, hardware, or computer applications that can provide a medium to exchange signals or data. The network 602 may be a local, regional, or global communication network. Various servers 601, such as a remote VCS Internet server, and associated database memory can connect with the at least a user device (1 . . . n). Additionally, various user devices (e.g. Smartphones, tablet computers, laptop computers, desktop computers and the like) can also connect to both the processor-controlled IoT hubs, sensors disposed on the device configured for data gathering, and/or the remote VCS Internet server 602.

As will be discussed, often a plurality of different user devices may be used, but for simplicity this plurality of devices will often be spoken of in the singular form. This use of the singular form is not intended to be limiting, and in general the claims and invention should be understood as operating with a plurality of devices. Although for simplicity, often mobile client computerized devices such as Internet connected versions of the popular Android, iOS, or Windows smartphones and tablets will be used as specific examples of devices, these specific examples are not intended to be limiting. The electronic computing device may include any number of sensors or components configured to intake or gather data from a user of the electronic computing device including, but not limited to, a camera, a heart rate monitor, a temperature sensor, an accelerometer, a microphone, and a gyroscope. The electronic computing device can also include an input device (e.g., a touchscreen or a keyboard) through which a user may input text and commands.

While not shown, note that server, Internet connected storage device and database memory may all be in the cloud. This is intended to both designate and remind the reader that the server, Internet connected storage device and database memory are in fact operating according to scalable Internet cloud-based methods that in turn operate according to automated service provisioning and automated virtual machine migration methods. As previously discussed, examples of such scalable methods include, but are not limited to, Amazon EC2, Microsoft Windows Azure platform, and the Google App

Engine. Thus, for example, server and Internet connected storage device will often be implemented as automatically provisioned virtual machines under a cloud service system that can create a greater or lesser number of copies of server and Internet connected video storage device and associated database memory according to the underlying demands on the system at any given time.

Preferred embodiments may include the addition of a remote server 601 or cloud server to further provide for back-end functionality and support. Any one of the storage or processing may be done on-board the device or be situated adjacent or remotely from the system and connected to each system via a communication network 601. In one embodiment, the server 601 may be used to support user behavior profiling; user history function; predictive learning/analytics; alert function; network sharing function; digital footprint tracking, etc. The remote server 601 may be further configured to authenticate the user and retrieve data of the user, device, and, or network and applies the data against a library of parameters, content, validated user information, etc.

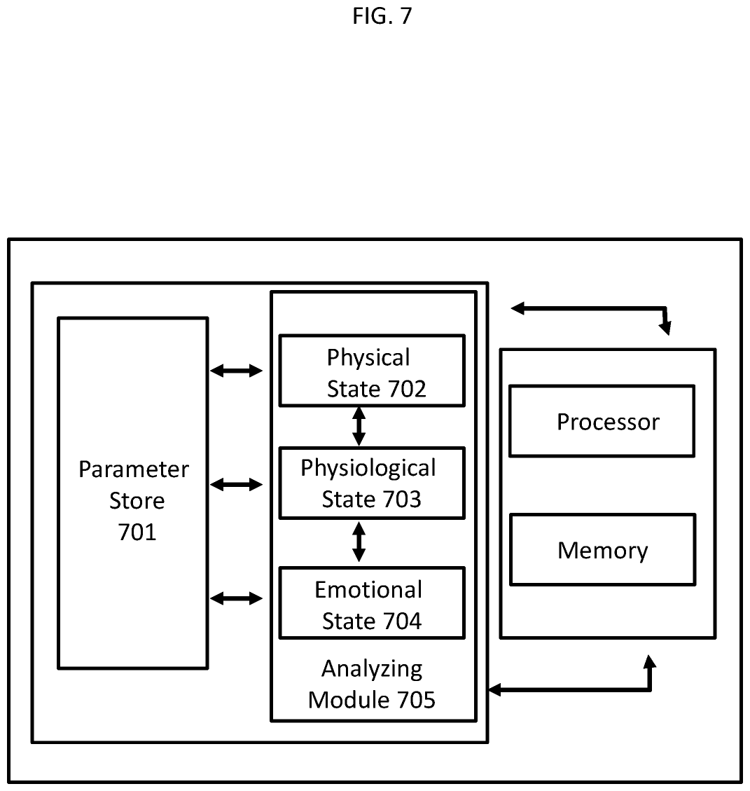

Now in reference to FIG. 7. FIG. 7 illustrate an exemplary embodiment of the flow-control breast-feeding system. FIG. 7 illustrates an exemplary processing unit with at least one physical state 702, physiological state 703, emotional state 704 configures for displaying interactively simulating flow control from the parameter store 701 based on a user-specific state. As shown, the system may comprise of an analyzing module 705 which determines a physical state 702, physiological state 703 or the emotional state 704 of the infant/user by mapping the received input parameter with a pre-stored set of parameters from the parameter store 701 to dynamically interact with the infant/user based on a determined output to promote a non-latching simulating breastfeeding experience to prevent bruised, sore nipples or engorged breasts.

As shown in FIG. 7 any number of physical state 701, physiological state 702 or emotional state 703 may be included in the parameter store 701. Each physical state 701, physiological state 702 or emotional state 703 may indicate at least one of a feeling, sensation, type of discomfort, mood, mental state, emotional condition, physical status of the user and, or a behavioral intervention or training regimen. FIG. 7 also illustrates the fact that any number of parameters may be associated with each physical state 701, physiological state 702 or emotional state 703. The matching of the parameters with the physical state 701, physiological state 702 or emotional state 703 may be pre-defined by at least one of an accredited expert or source; probabilistic; or deep learned. In a preferred embodiment, an accredited expert or source will require at least two independent sources of peer-reviewed scholarship or data to validate the match.

The analyzing module 705 may push a parameter to the user based on at least one stored parameter matched to the selected physical state 701, physiological state 702 or emotional state 703 of the user. For example, within the parameter store 701, if parameter 1 (fussy) is selected as defined by the user or the system, any one of parameter 1, 2 . . . n may be selected by the analyzing module 705. The pre-defined parameters validated by the accredited expert may all be parameters with documented utility in elevating mood and energy (rubric). The mood and energy documented for each parameter may be on a scale. For instance, parameter 1 may be low-moderate; parameter 2 may be moderate; and parameter n may be high-severe, etc. Any variant of the scale may be featured without departing from the scope of the invention. In other embodiments, the parameters, while falling under the same rubric and un-scaled, can vary along design cues. Other user profile or demographic information may further inform the analyzing module 705 choice of parameter type, such as age, ethnicity, etc. User profile or demographic information may be user inputted or digitally crawled.

Still in reference to FIG. 7, the analyzing module 701 choice of parameter type is not specific to a user, user profile, or crawled user data. In a certain embodiment, the prescriber 305 may have to choose between any one of the parameter types (parameter 1, parameter 2 . . . parameter n) from the selected physical, emotional, or physiological type. This type of parameter assignment may be completely arbitrary. In other embodiments, the parameter assignment may be not specific to a user-generated or crawled profile but may be based on user history. In other words, a user's tracked level of engagement with a previous parameter or parameters from a previous session may inform parameter assignment by the analyzing module 701. Tracking engagement of a user with a pushed parameter may be by camera-captured eye gazing, touch-screen interaction, time span between pushed message and user follow-up action, choice of follow-up action, etc.

In some embodiments, the full list of parameter types is not grouped by physical, physiological, or emotional type or along any design categories, but rather simply listed arbitrarily and mapped or matched to an appropriate physical, physiological or emotional type. In this arbitrarily listed manner, the analyzing module 705 may match to more than one physical, physiological, or emotional type. Likewise, a user may be defined by more than one physical, physiological or emotion type and be analyzed as to be having the same parameter type.

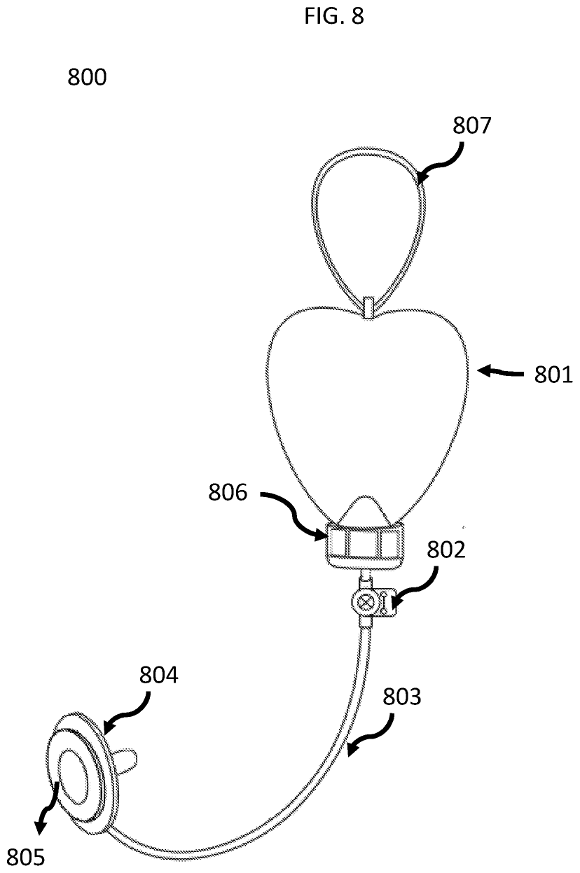

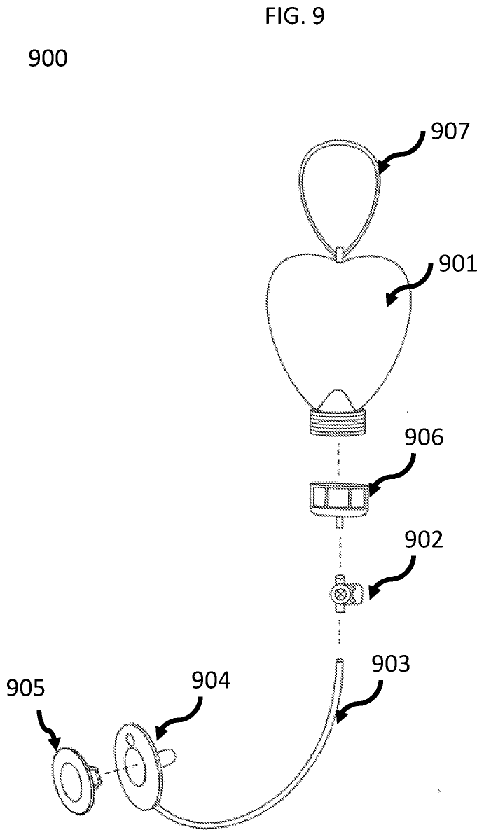

Now in reference to FIGS. 8 and 9. In an embodiment of the invention, a simulating flow control breast-feeding system 800, 900 comprising of a tube 803, 903 linking a refillable housing chamber 801, 901 to a prosthetic nipple 804, 904, an analyzing module linked to a sensor 802, 902, a network interface, a non-transitory storage element coupled to a processor and encoded instructions stored in the non-transitory storage element, wherein the encoded instructions when implemented by the processor, configure the system to receive an input parameter from an infant/user to determine a physical, physiological, or emotional state of the infant/user by mapping the received input parameter with a pre-stored set of parameters from the parameter store via the analyzing module and dynamically interact with the infant/user based on a determined output to promote a non-latching simulating breastfeeding experience to prevent bruised, sore nipples or engorged breasts.

Further yet, in an embodiment of the invention, the sensor 802, 902 is attached to at least one of, sealing contraption 806, 906, strap 807, 907, prosthetic nipple 804, 904, tubing 803, 903 or the refillable housing chamber 801, 901. Additionally, the sealing contraption 806, 906, strap 807, 907, prosthetic nipple 804, 904, tubing 803, 903 or the refillable housing chamber 801, 901 is modular. These modular components make up a functioning simulating flow control breast-feeding system.

In yet another embodiment of the invention, the sensor 802, 902 is at least one of, a motion sensor, an accelerometer, a temperature gauge, a 3D accelerometer, a gyroscope, a pressure sensor, a global positioning system sensor (GPS), a magnetometer, an inclinometer, and an impact sensor. Additionally, in an embodiment of the invention, the system 800, 900 may further comprise of a sensor 802, 902 attached to at least one of, but not limited to, a sealing contraption 806, 906, neck strap 807, 907, prosthetic nipple 804, 904, tubing 803, 903 or the refillable housing 801, 901. To enable continuous monitoring, the user uses a sensor 802, 902 attached on any one of, but not limited to, the sealing contraption 806, 906, neck strap 807, 907, prosthetic nipple 804, 904, tubing 803, 903 or the refillable housing chamber 801, 901 and a mobile communication device (not shown). The sealing contraption 802, 902, neck strap 807, 907, prosthetic nipple 804, 904, tubing 803, 903 or the refillable housing chamber 801, 901 are typically embedded/equipped with one or more motion sensors, physiological sensors and environmental sensors. Examples of these sensors include, but are not limited to accelerometers, gyroscopes, inclinometers, geomagnetic sensors, global positioning systems, impact sensors, microphones, cameras, heart rate monitors, pulse oximeters, blood alcohol monitors, respiratory rate sensors, transdermal sensors, galvanic skin response (GSR) sensors and electromyography (EMG) sensors.

Further yet, the simulating system 800, 900 may be worn on one or more body parts of the user, such as wrist, waist, neck, arm, leg, abdomen, chest, thigh, head, ear and fingers as well as attached to a plurality of attachments such as, but not limited to, an armband, headband, backpack worn by the user.

The mobile communication device is a portable device that has the capability of communicating over the network in real time, presenting periodic surveys to the user and receiving response from the infant/user on the periodic surveys. Examples of the mobile communication device include, but are not limited to, a smartphone, a tablet, a personal digital assistant (PDA) and a mobile phone.

Further yet in another embodiment of the invention, a simulating flow control breast-feeding system comprising of a snap-fit tube (not shown) linking a refillable housing chamber 801, 901 to a prosthetic nipple 804, 904 via a sealing contraption 806, 906 wherein, the prosthetic nipple 804, 904 comprises of an opening to deliver the fluid from the refillable housing chamber 801, 901 to an infant/user, a network interface, an analyzing module coupled to a sensor 802, 902, a non-transitory storage element coupled to a processor and encoded instructions stored in the non-transitory storage element, wherein the encoded instructions when implemented by the processor, configure the system to receive an input parameter from an infant/user and determine a physical, physiological or emotional state of the infant/user by mapping the received input parameter with a pre-stored set of parameters via the analyzing module and dynamically interact with the infant/user based on a determined output to promote a non-latching simulating breastfeeding experience to prevent bruised, sore nipples or engorged breasts.

In yet another embodiment of the invention, the flow control breast-feeding system further comprises of a detachable plug 805, 905 to create an air gap, connected to the prosthetic nipple 804, 904 to avoid a backflow of the fluid into refillable housing chamber 801, 901. Additionally, the refillable housing chamber 801, 901, prosthetic nipple 804, 904, detachable plug 805, 905, or the tubing 803, 903 is at least one of, a plurality of shapes, sizes, colors or opaqueness and the various parts are modular as well. Further yet, the modular parts may be made of at least one of, non-GMO, non-toxic, organic-grade glue, BPA free or hypoallergic material.

Further yet, in a preferred embodiment of the invention, the prosthetic nipple 804, 904, detachable plug 805, 905 or the tubing 803, 903 comprises of, at least one of, naturally or man-made rubber, synthetic rubber, silicone or any plastic or synthetic polymer. In addition, the back end of the prosthetic nipple 804, 904 maybe attachable either via the detachable plug 805, 905 or directly adhered to the skin via a self-adhering glue to establish a tight seal. Additionally, the size of the prosthetic nipple 804, 904 maybe directly proportional to any one of age, gender, weight, food intake of the infant.

In yet another embodiment of the invention, a simulating flow control breast-feeding system comprising of a strap fastened to a refillable housing chamber 801, 901, a sealing contraption 806, 906 attached to the refillable housing chamber 801, 901, a snap-fit tube linking the refillable housing chamber 801, 901 to a prosthetic nipple 804, 904 wherein, the prosthetic nipple 804, 904 comprises of an opening to deliver the fluid from the refillable housing chamber 801, 901 to an infant/user, a network interface, an analyzing module coupled to a sensor 802, 902, a non-transitory storage element coupled to a processor and, encoded instructions stored in the non-transitory storage element, wherein the encoded instructions when implemented by the processor, configure the system to receive an input parameter from an infant/user, determine a physical, physiological or emotional state of the infant/user by mapping the received input parameter with a pre-stored set of parameters via the analyzing module and dynamically interact with the infant/user based on a determined output to promote a non-latching simulating breastfeeding experience to prevent bruised, sore nipples or engorged breasts. In an embodiment of the invention, a clamp (not shown) may be fitted on the tubing to control the flow of the fluid from the refillable housing chamber to the prosthetic nipple.

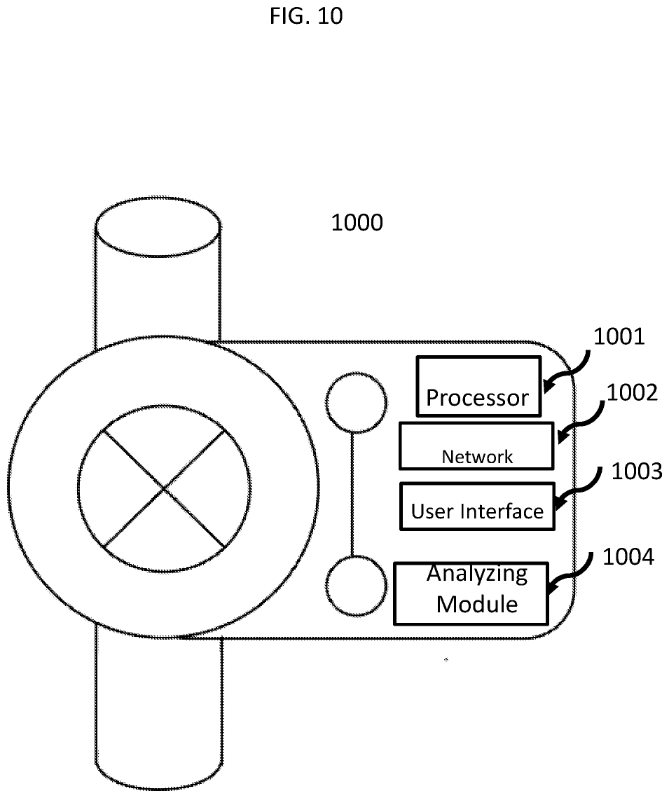

Now in reference to FIG. 10, in an embodiment of the present invention, the data captured by the one or more sensors 1000 of the simulating flow control breast-feeding system is first sent to the mobile communication device and thereby, sent to the processing unit/processor 1001 over the network. The simulating breast-feeding system communicates with the mobile communication device over a short-range wireless communication medium. Examples of the short-range wireless communication medium include Bluetooth, ZigBee, Infrared, Near Field Communication (NFC) and Radio-frequency identification (RFID).

The network 1002 may be any suitable wired network, wireless network, a combination of these or any other conventional network, without limiting the scope of the present invention. Few examples may include a LAN or wireless LAN connection, an

Internet connection, a point-to-point connection, or other network connection and combinations thereof. The network 1002 may be any other type of network that is capable of transmitting or receiving data to/from host computers, personal devices, telephones, video/image capturing devices, video/image servers, or any other electronic devices. Further, the network 1002 is capable of transmitting/sending data between the mentioned devices. Additionally, the network 1002 may be a local, regional, or global communication network, for example, an enterprise telecommunication network, the Internet, a global mobile communication network, or any combination of similar networks. The network 1002 may be a combination of an enterprise network (or the

Internet) and a cellular network, in which case, suitable systems and methods are employed to seamlessly communicate between the two networks. In such cases, a mobile switching gateway may be utilized to communicate with a computer network gateway to pass data between the two networks. The network 1002 may include any software, hardware, or computer applications that can provide a medium to exchange signals or data in any of the formats known in the art, related art, or developed later. Further yet, the sensor may include a user interface 1003.

In an embodiment of the present invention, the data captured by the one or more sensors 1000 is sent to the processing unit/processor 1001 through the network 1002. Further yet, the simulating breast-feeding system may comprise of a sensor 1000 to monitor fluid consumption to gauge if, the infant is feeding for comfort or hunger. Additionally, in another embodiment of the invention, the simulating breast-feeding system may comprise sensors 1000 to detect at least one of, but not limited to, infant temperature, heart rate, pulse rate, respiratory patterns or rhythms or accuracy of latching.

In an embodiment of the present invention, the processing unit/processor 1001 receives the sensor data in the form of the input parameter from the simulating breast-feeding system 1000 as well as responses on the periodic surveys from the user/infant. Thereby, the processing unit/processor 1001 correlates the sensor data with the responses on periodic surveys, generates reports corresponding to the physical, physiological or emotional state of the user/infant via the analyzing module 1004 and sends the reports and other relevant data to the user. These reports enable the user to track/monitor the progression of feeding in the infant. In an embodiment of the present invention, the user is enabled to view the reports, as generated by the processing unit/processor 1001 using one or more devices selected from the group comprising a smartphone, a computer, a laptop, a tablet, a personal digital assistant (PDA) and a mobile phone. Additionally, in an embodiment of the invention, the input parameter may be at least one of, a motion, sleep, gestural, visual, audio, or environmental condition.

The pre-stored of parameters may be a plurality of infant behaviors as outlined in

American academy of Pediatrics, World health organization, National Institute of child health and human development, Food and Drug administration, American academy of family physicians. Further yet, in another embodiment of the invention, the physical input parameter may be at least one of, but not limited to, motion, sleep, gestural, visual, audio or environmental factors/conditions. Moreover, the sleep related characteristics of the infant may be indicative of at least one of, duration of sleep time, number of times awake, sound sleep, light sleep and awake time. Additionally, the physiological input parameters may be at least one of, but not limited to, age, gender, race, medical history, heart rate, medication history, blood pressure, sweat, duration of feeding, number of feedings and food intake of user/infant.

Additionally, environmental conditions may affect infant activity. The environmental conditions can be at least one of, but not limited to, wind velocity, temperature, humidity, aridness, light, darkness, noise pollution, exposure to UV, airborne pollution and radioactivity. Further yet, data generated from a set of parameters corresponding to at least one of, but not limited to, user/infant reported symptoms and side effects, periodic surveys may be used to generate a personal profile of the infant/user via the analyzing module 1004. The data generated may in any one of, but not limited to, audio, video or an image input and further, implemented on at least one of, but not limited to a mobile communication device, body worn device, wearable device, tablet and or IoT. Further yet, in an embodiment wherein the pre-stored set of parameters is at least one of, cry, hiccup, sneeze, yawn, burp and gurgle of the infant/user.

Further yet, in an embodiment of the invention, a deviation from the personal profile may be alerted to the user by at least one of, a visual or color appearance, audio or sound, buzzer, vibration, fine motor skills, memory-based tasks, repeating actions, sounds or movements. Moreover, the simulating breast-feeding system may comprise detection a deviation from the personal profile of the user/infant using machine learning algorithm.

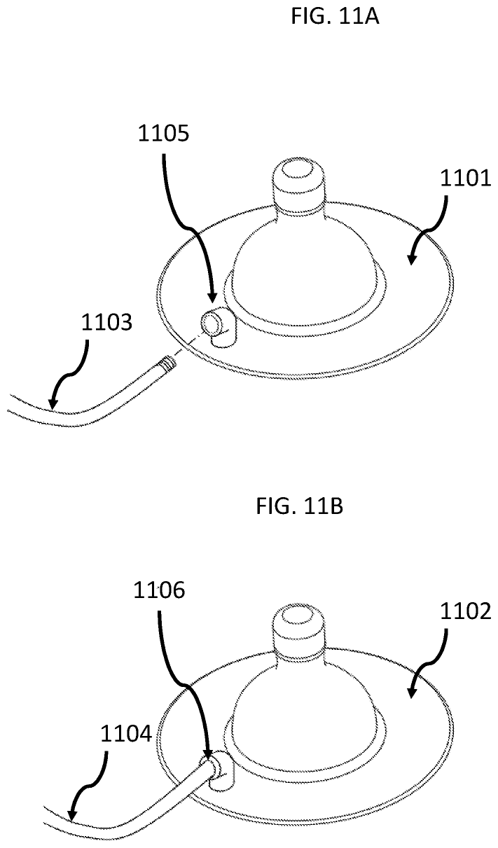

Now in reference to FIG. 11A and FIG. 11B. In an embodiment of the present invention, the tube 1103, 1104 links the prosthetic nipple 1101, 1102 via a snap-fit mechanism 1105, 1106 to prevent leakage and, or back flow of the fluid from the prosthetic nipple 1101, 1102 to the refillable housing chamber (not shown) via the tube 1103, 1104. In yet another embodiment of the invention, the tube 1103, 1104 is connected to the prosthetic nipple 1101, 1102 by an alternate mechanism, for example, a rubber seal, push-in mechanism.

Embodiments are described at least in part herein with reference to flowchart illustrations and/or block diagrams of methods, systems, and computer program products and data structures according to embodiments of the disclosure. It will be understood that each block of the illustrations, and combinations of blocks, can be implemented by computer program instructions. These computer program instructions may be provided to a processor of a general-purpose computer, special purpose computer, or other programmable data processing apparatus to produce a machine such that the instructions, which execute via the processor of the computer or other programmable data processing apparatus, create means for implementing the functions/acts specified in the block or blocks.

These computer program instructions may also be stored in a computer-readable memory that can direct a computer or other programmable data processing apparatus to function in a particular manner such that the instructions stored in the computer-readable memory produce an article of manufacture including instruction means which implement the function/act specified in the block or blocks.

The computer program instructions may also be loaded onto a computer or other programmable data processing apparatus to cause a series of operational steps to be performed on the computer or other programmable apparatus, to produce a computer implemented process such that, the instructions which execute on the computer or other programmable apparatus provide steps for implementing the functions/acts specified in the block or blocks.

In general, the word “module” as used herein, refers to logic embodied in hardware or firmware, or to a collection of software instructions, written in a programming language, such as, Java, C, etc. One or more software instructions in the unit may be embedded in firmware. The modules described herein may be implemented as either software and/or hardware modules and may be stored in any type of non-transitory computer-readable medium or other non-transitory storage elements. Some non-limiting examples of non-transitory computer-readable media include CDs, DVDs, BLU-RAY, flash memory, and hard disk drives.

In the drawings and specification, there have been disclosed exemplary embodiments of the disclosure. Although specific terms are employed, they are used in a generic and descriptive sense only and not for purposes of limitation, the scope of the invention being defined by the following claims. Those skilled in the art will recognize that the present invention admits of a number of modifications, within the spirit and scope of the inventive concepts, and that it may be applied in numerous applications, only some of which have been described herein. It is intended by the following claims to claim all such modifications and variations which fall within the true scope of the invention.

1. A simulating flow control breast-feeding system comprising:
a tube linking a refillable housing chamber to a prosthetic nipple;
an analyzing module linked to a sensor;
a network interface;
a non-transitory storage element coupled to a processor and;
encoded instructions stored in the non-transitory storage element, wherein the encoded instructions when implemented by the processor, configure the system to:
receive an input parameter from an infant/user;
determine a physical, physiological, or emotional state of the infant/user by mapping the received input parameter with a pre-stored set of parameters from the parameter store via the analyzing module; and
dynamically interact with the infant/user based on a determined output to promote a non-latching simulating breastfeeding experience to prevent bruised, sore nipples or engorged breasts.

2. The system of claim 1, wherein the sensor is attached to at least one of, sealing contraption, strap, prosthetic nipple, tubing or the refillable housing chamber.

3. The system of claim 1, wherein at least one of the sealing contraption, strap, prosthetic nipple, tubing or the refillable housing chamber is modular.

4. The system of claim 2, wherein the sensor is at least one of, a motion sensor, an accelerometer, a temperature gauge, a 3D accelerometer, a gyroscope, a pressure sensor, a global positioning system sensor (GPS), a magnetometer, an inclinometer, and an impact sensor.

5. The system in claim 2, wherein the sensor monitors fluid consumption or flow to evaluate if the infant is feeding for comfort or hunger.

6. The system in claim 2, wherein the sensor detects at least one of, infant temperature, heart rate, pulse rate, respiratory patterns or rhythms or accuracy of latching.

7. The system of claim 1, wherein the input parameter may be at least one of, a motion, sleep, gestural, visual, audio, or environmental condition.

8. The system of claim 1, further comprising establishing a personal profile of the infant/user based on at least one set of parameters via the analyzing module.

9. The system of claim 1, wherein the pre-stored set of parameters is at least one of, cry, hiccup, sneeze, yawn, burp and gurgle of the infant/user.

10. The system of claim 1, wherein deviation from the personal profile detected by the analyzing module may be alerted to the user by, at least one of, a visual or color appearance, audio or sound, buzzer, vibration, fine motor skills, memory-based tasks, repeating actions, sounds or movements.

11. The system of claim 1, further comprising detecting a deviation from the personal profile of the infant/user using machine learning algorithm.

12. A simulating flow control breast-feeding system comprising:
a snap-fit tube linking a refillable housing chamber to a prosthetic nipple via a sealing contraption wherein, the prosthetic nipple comprises of an opening to deliver the fluid from the refillable housing chamber to an infant/user;
a network interface;
an analyzing module coupled to a sensor;
a non-transitory storage element coupled to a processor and;
encoded instructions stored in the non-transitory storage element, wherein the encoded instructions when implemented by the processor, configure the system to:
receive an input parameter from an infant/user;
determine a physical, physiological or emotional state of the infant/user by mapping the received input parameter with a pre-stored set of parameters via the analyzing module; and
dynamically interact with the infant/user based on a determined output to promote a non-latching simulating breastfeeding experience to prevent bruised, sore nipples or engorged breasts.

13. The system of claim 12, further comprising a detachable plug to create an air gap, connected to the prosthetic nipple to avoid a backflow of the fluid into refillable housing chamber.

14. The system of claim 12, wherein the refillable housing chamber, prosthetic nipple, detachable plug or the tubing is at least one of, a plurality of shapes, sizes, colors or opaqueness.

15. The system of claim 12, further comprising at least one of, strap, refillable housing chamber, sealing contraption, tubing, detachable plug or prosthetic nipple be modular.

16. The system of claim 12, wherein the modular part may be made of at least one of, non-GMO, non-toxic, organic-grade glue, BPA free or hypoallergic material.

17. The system of claim 12, wherein the prosthetic nipple, detachable plug or the tubing comprises of, at least one of, naturally or man-made rubber, synthetic rubber, silicone or any plastic or synthetic polymer.

18. The system of claim 12, wherein a back end of the prosthetic nipple maybe attachable either via the detachable plug or directly to the skin via a self-adhering glue to establish a tight seal.

19. The system of claim 12, wherein a size of the prosthetic nipple maybe directly proportional to any one of age, gender, weight, food intake of the infant.

20. A simulating flow control breast-feeding system comprising:
a strap fastened to a refillable housing chamber;
a sealing contraption attached to the refillable housing chamber;
a snap-fit tube linking the refillable housing chamber to a prosthetic nipple wherein, the prosthetic nipple comprises of an opening to deliver the fluid from the refillable housing chamber to an infant/user;
a network interface;
an analyzing module coupled to a sensor;
a non-transitory storage element coupled to a processor and;
encoded instructions stored in the non-transitory storage element, wherein the encoded instructions when implemented by the processor, configure the system to:
receive an input parameter from an infant/user;
determine a physical, physiological or emotional state of the infant/user by mapping the received input parameter with a pre-stored set of parameters via the analyzing module; and
dynamically interact with the infant/user based on a determined output to promote a non-latching simulating breastfeeding experience to prevent bruised, sore nipples or engorged breasts.
